(19) United States
(12) Patent Application Publication
(10) Pub. No.: US 2014/0323322 A1
(43) Pub. Date: Oct. 30, 2014

(54) METHOD AND APPARATUS FOR CHEMICAL SENSING USING 2D PHOTONIC CRYSTAL ARRAYS

(71) Applicant: Sanford A. Asher, Pittsburgh, PA (US)

(72) Inventor: Sanford A. Asher, Pittsburgh, PA (US)

(21) Appl. No.: 13/645,458

(22) Filed: Oct. 4, 2012

Related U.S. Application Data
(60) Provisional application No. 61/543,000, filed on Oct. 4, 2011.

(51) Int. Cl. G01N 33/543; C12Q 1/62; G01N 33/84; G01N 33/52; G01N 33/50

(52) U.S. Cl. CPC G01N 33/54373; G01N 33/525; G01N 33/5038; G01N 33/84; C12Q 1/62
USPC 506/9; 422/82.05; 422/69; 435/287.7; 422/82.06; 436/169; 435/29; 436/74; 436/86; 436/501; 436/87; 436/113; 436/163; 435/25; 422/422; 506/15; 506/32; 506/12; 506/18; 156/307.1

(57) ABSTRACT

A chemical sensor comprising: a hydrogel layer, comprising one or more molecular recognition agents and a 2DPC self-assembling array; and a mirror layer. A method for analyzing a sample or bodily fluid, comprising: obtaining a sample or bodily fluid; placing an amount of the sample or bodily fluid onto a chemical sensor, comprising: a hydrogel layer, comprising a molecular recognition agent and a 2DPC self-assembling array; a tethering hydrogel layer; a mirror layer; and a membrane filter layer, allowing the bodily fluid to interact with the hydrogel layer; and allowing ambient or artificial light to pass through the hydrogel layer onto the mirror layer and observing a change in diffraction versus a control.

### RELATED APPLICATION

This application claims priority benefit under 35 U.S.C. §119(e) of U.S. Provisional Application No. 61/543,000, filed Oct. 4, 2011, the contents of which are herein incorporated by reference.

### GOVERNMENTAL RIGHTS

This invention was made with government support under HDTRA1-10-1-0044 awarded by the Defense Threat Reduction Agency (DTRA). The government has certain rights in the invention.

### TECHNICAL FIELD

The present disclosure relates to novel chemical sensors that can be used for real time, point-of-care sensing of clinically important analytes. The novel chemical sensors of the present disclosure utilize novel nanoscale, chemically sensitive, self-assembling, two-dimensional photonic crystal (2DPC) arrays hydrogels. Additionally, the present disclosure describes processes for the preparation of novel chemical sensors that can be used for real time, point-of-care sensing of clinically important analytes. Also, the present disclosure describes the use of the novel chemical sensors in the analyses of bodily fluids.

### BACKGROUND

Diagnosis of disease is the first essential step in the path to relieve human illness. Many of the recent advances in medicine result from advances in the understanding of the biochemical basis of disease, and from the development of new clinical chemistry techniques to monitor analytes that report on the disease state. Multiple analytical methodologies are used to meet the broad range of analyses requested by today's physicians. Almost all of these methodologies are fully concentrated in the central clinical laboratory and thus much inefficiency exists. The current United States in vitro testing market exceeds $21.4 billion and growing 6% annually.

The volume of tests performed in large hospital clinical laboratories for basic metabolic profiles (Na⁺, K⁺, Cl⁻, BUN, etc.) may be greater that 400,000 per year per test. These basic metabolic analyses are the initial routine diagnostic investigations performed for the majority of hospital admissions. Automated testing systems are the mainstay of analysis because of the resulting high volumes. A large number of different analytical platforms are used within each clinical laboratory. Some of these platforms serially accomplish individual tests, while others accomplish numerous tests in parallel by utilizing a variety of sophisticated liquid chemistries and/or highly automated dry multilayer film high throughput tests. All of these tests are expensive and require highly sophisticated equipment that is not amenable fore use at the bedside.

Hospital based point-of-care systems are already used for diagnosis and treatment. Multi-functional systems have been developed for interfacing point-of-care instrumentation with laboratory information management systems both wired and wireless. Multi-modal instruments are also available. Units such as developed by i-STAT Corporation provide bedside analysis for common analytes such as Na⁺, K⁺, Cl⁻, Ca²⁺, glucose, pH, pCO₂, urea, etc. Their instruments utilize traditional electrochemistry combined with thin film microfabricated electrodes.

Point-of-care devices are utilized throughout healthcare not just in hospitals. Home glucose monitoring is now the standard of care for millions of patients with diabetes. Patients with nephrosis and other kidney diseases monitor urine protein at home. Home pregnancy testing is commonplace and on-site testing for drugs of abuse and blood cholesterol is also available.

The 2DPC sensors of the present disclosure work well when exposed to whole blood since they will detect only the extracellular species over the short times of analysis. The intracellular milieu of the red cell will effectively be compartmentalized away from the active components of the 2DPC sensors. Hence, the values determined will be comparable to plasma samples. The 2DPC point-of-care sensors of the present disclosure are especially useful in the assessment of a patient's condition on-site following traumatic injuries, where time is of the essence in treatment. The 2DPC point-of-care sensors of the present disclosure thus also make effective point-of-care clinical chemistry a valuable adjunct to medical decision making during the critical transport time between the site of injury and the hospital.

Therefore, a real time, point-of-care chemical sensor would both serve to relieve human illness and be financially beneficial. These and other developments are a result of the present disclosure.

### SUMMARY

In a preferred aspect, the present disclosure is directed to novel visible and near IR wavelength diffracting 2DPC arrays in hydrogels that report on the concentration of clinically important analytes. The 2DPC array sensor response is actuated by molecular recognition agents and by hydrogel cavities imprinted by the targeted analytes.

In another preferred aspect, the present disclosure is directed to novel ultrasensitive 2DPC array sensors that are highly elastic such that they undergo large 2DPC array spacing changes in response to contact with low concentrations of analytes.

In yet another preferred aspect, the present disclosure is directed to novel 2DPC point-of-care sensors that monitor macromolecular interactions such as antigen-antibody binding. Preferably, the 2DPC sensors of the present disclosure are utilized to determine analytes on cell surfaces in cell culture and on tissue surfaces for use in biomolecular studies that correlate cell response to drugs and chemical environmental challenges; measuring the extracellular pH of polarized apical proton-secreting cell lines in culture; and/or studying of acid-base membrane transport proteins in response to pharmacologic agonists or in response to knock-down or overexpression of transport proteins and/or their regulators.

In a preferred aspect, the present disclosure is directed to a chemical sensor comprising:
a) a hydrogel layer, comprising one or more molecular recognition agents and a 2DPC self-assembling array; and
b) a mirror layer.

In another preferred aspect, the present disclosure is more specifically directed to a chemical sensor, additionally comprising a tethering hydrogel layer.

In yet another preferred aspect, the present disclosure is more specifically directed to a chemical sensor, additionally comprising a membrane filter layer,

In another preferred aspect, the present disclosure is more specifically directed to a chemical sensor wherein, the layers are arranged such that an amount of the sample or bodily fluid to be analyzed may be placed on an exposed surface of the membrane filter layer, travel through the membrane filter layer, the mirror layer and the tethering hydrogel layer to the hydrogel layer.

In yet a further preferred aspect, the present disclosure is more specifically directed to a chemical sensor, wherein: the mirror layer is selected from the group consisting of aluminum and silver.

In another preferred aspect, the present disclosure is more specifically directed to a chemical sensor, wherein the membrane filter layer is designed to filter blood cells and allow blood plasma to pass through to the mirror layer.

In an additional preferred aspect, the present disclosure is more specifically directed to a chemical sensor, wherein the tethering hydrogel layer contains a low density of hydrolysis resistant crosslinkers.

In yet another preferred aspect, the present disclosure is more specifically directed to a chemical sensor, wherein the hydrolysis resistant crosslinkers are long-chain PEG-type crosslinkers.

In another preferred aspect, the present disclosure is more specifically directed to a chemical sensor, wherein the hydrogel layer has a very low crosslink density.

In yet another preferred aspect, the present disclosure is more specifically directed to a chemical sensor, wherein the hydrogel layer is formulated to perform an analysis selected from the group consisting of: blood osmolality quantitation; blood pH determination; cation detection and/or quantitation; anion detection and/or quantitation; ammonia detection and/or quantitation, metal detection and/or quantitation; urea detection and/or quantitation; uric acid detection and/or quantitation; protein detection and/or quantitation; and cell and tissue surface chemical detection and/or quantitation.

In another preferred aspect, the present disclosure is more specifically directed to a chemical sensor, wherein the hydrogel layer contains an antibody or a monoclonal antibody.

In yet another preferred aspect, the present disclosure is more specifically directed to a chemical sensor, wherein the cation to be detected and/or quantified is selected from the group consisting of: sodium, potassium, calcium, magnesium, ammonium and zinc.

In another preferred aspect, the present disclosure is more specifically directed to a chemical sensor, wherein the anion to be detected and/or quantified is selected from the group consisting of: chloride, phosphate and bicarbonate.

In a further preferred aspect, the present disclosure is more specifically directed to a chemical sensor, wherein the metal to be detected and/or quantified is mercury.

In another preferred aspect, the present disclosure is more specifically directed to a chemical sensor further comprising a wick for conveying the sample or bodily fluid to a plurality of different molecular recognition agents in the hydrogel layer.

In yet another preferred aspect, the present disclosure is more specifically directed to a chemical sensor, wherein the chemical sensor has an inlet for a sample or bodily fluid and a wick for conveying the sample or bodily fluid to the membrane filter layer.

In another preferred aspect, the present disclosure is more specifically directed to a chemical sensor, wherein the wick conveys the sample or bodily fluid to multiple membrane filter layers simultaneously.

In yet a further preferred aspect, the present disclosure is directed to a process for the preparation of a chemical sensor comprising:
a hydrogel layer, comprising a molecular recognition agent and a 2DPC self-assembling array;
a tethering hydrogel layer;
a mirror layer; and
a membrane filter layer,

comprising:
chemically attaching the mirror layer to the membrane filter layer:
preparing the mirror layer for polymerization;
polymerizing the tethering hydrogel layer, with a high density of easily broken crosslinks and a lower density of chemically stable long-chain tethering crosslinks, onto the prepared mirror layer;
polymerizing a hydrogel layer, comprising molecular recognition agent and a 2DPC self-assembling array onto the tethering hydrogel layer; and
hydrolyzing the easily broken crosslinks in the tethering hydrogel layer.

In another preferred aspect, the present disclosure is directed to the preparation of a chemical sensor wherein the polymerizing and hydrolyzing are reversed in order of performance.

In yet another preferred aspect, the present disclosure is more specifically directed to a process for the preparation of a chemical sensor, wherein the mirror layer is prepared with one or more vinyl groups.

In yet a further preferred aspect, the present disclosure is more specifically directed to a process for the preparation of a chemical sensor, wherein the easily broken crosslinks have an ester functionality.

In yet another preferred aspect, the present disclosure is more specifically directed to a process for the preparation of a chemical sensor, wherein the chemically-stable long-chain tethering crosslinks have an amide functionality.

In another preferred aspect, the present disclosure is more specifically directed to a process for the preparation of a chemical sensor, wherein the ester functionalities are hydrolyzed under basic conditions in the presence of tetramethyehtylenediamine (TEMED).

In yet an additional preferred aspect, the present disclosure is more specifically directed to a process for the preparation of a chemical sensor, wherein the hydrogel layer, comprising a molecular recognition agent and a 2DPC self-assembling array is less than about 1 μM thick.

In yet another preferred aspect, the present disclosure is directed to a method for analyzing a sample or bodily fluid, comprising:
obtaining a sample or bodily fluid;
placing an amount of the sample or bodily fluid onto a chemical sensor, comprising:
   a hydrogel layer, comprising a molecular recognition agent and a 2DPC self-assembling array;
   a tethering hydrogel layer;
   a mirror layer; and
   a membrane filter layer,
allowing the sample or bodily fluid to interact with the hydrogel layer; and
allowing ambient or artificial light to pass through the hydrogel layer onto the mirror layer and observing the change in diffraction versus a control.

In another preferred aspect, the present disclosure is more specifically directed to the method of analyzing a sample or bodily fluid, wherein the change in diffraction is observed through the matching of the color to a color chart.

In yet another preferred aspect, the present disclosure is more specifically directed to the method of analyzing a sample or bodily fluid, wherein the change in diffraction is observed through the use of a reflectance probe spectrophotometer.

In yet another preferred aspect, the present disclosure is more specifically directed to the method of analyzing a sample or bodily fluid, wherein the artificial light may be provided by a light source from the group consisting of: lantern, flashlight, indoor lighting, camera flash and camera light.

In another preferred aspect, the present disclosure is more specifically directed to the method of analyzing a sample or bodily fluid, wherein the camera may be a charge-coupled device (CCD) camera/light source combination, such as, for example, a cell phone camera.

In yet another preferred aspect, the present disclosure is more specifically directed to the method of analyzing a sample or bodily fluid, wherein the CCD camera/light source combination:

a) records at least one photograph of the hydrogel layer; and

b) analyzes the photograph against stored calibration data.

In another preferred aspect, the present disclosure is more specifically directed to the method of analyzing a sample or bodily fluid, wherein the analyses may be time dependent and a series of photographs are taken and analyzed.

In yet a further preferred aspect, the present disclosure is more specifically directed to a method for analyzing a sample or bodily fluid, wherein the chemical sensor has an inlet for the sample or bodily fluid and a wick for conveying the sample or bodily fluid to the membrane filter layer.

In yet another preferred aspect, the present disclosure is more specifically directed to a method for analyzing a sample or bodily fluid, wherein the wick conveys the sample or bodily fluid to multiple membrane filter layers simultaneously.

In another preferred aspect, the present disclosure is directed to an analytical kit for analyzing a sample or bodily fluid, comprising:

a chemical sensor, comprising:
a hydrogel layer, comprising molecular recognition agent and a 2DPC self assembling array;
a tethering hydrogel layer;
a mirror layer; and
a membrane filter layer,

a CCD camera/light source combination; and

diagnostic software.

In yet another preferred aspect, the present disclosure is more specifically directed to a chemical sensor comprising: an ultrathin hydrogel layer, comprising an imprinted analyte sensor and/or a molecular recognition agent; and a mirror layer; wherein the hydrogel layer diffracts about 80% of incident light.

In yet another preferred aspect, the present disclosure is more specifically directed to a chemical sensor wherein about 90% of incident light diffracted by the hydrogel layer is forward diffracted.

Having briefly described the present disclosure, the above features and advantages thereof will be recognized by those skilled in the pertinent art from the following detailed description.

### BRIEF DESCRIPTION OF THE DRAWINGS

FIG. 1a shows a hydrogel layer according to a preferred embodiment of the present disclosure comprising one or more molecular recognition agents and a 2DPC self-assembling array tethered to Al or Ag mirror by a tethering hydrogel layer that does not constrain volume changes of the sensing hydrogel. The mirror reflects back the forward diffracted light. Whole blood is analyzed preferably by introducing it from the bottom through the membrane filter layer. The membrane filter layer at the bottom excludes blood cells.

FIG. 1b illustrates bright diffracted blue light of 2DPC and red light of swollen 2DPC of about 500 nm polystyrene particle 2D array according to a preferred embodiment of the present disclosure.

FIG. 2 shows a preferred design of a point-of-care, two-dimensional photonic crystal (2DPC) sensor array according to a preferred aspect of the present disclosure for nine analytes. Fingerstick blood samples applied to the inlet are quickly wicked through the microfluidic channels to the hydrogel layer, comprising molecular recognition agent and 2DPC self-assembling array. The array elements each change their diffraction colors to indicate the different analyte concentrations.

FIG. 3 shows that according to a preferred aspect of the present disclosure, 2DPC array diffraction disperses the light over a range of angles, diffracting most of the incident light and that the forward diffraction is more intense than the back diffraction.

FIG. 4 shows a Littrow configuration reflection probe fiber-optic sensing of 2DPC diffraction according to a preferred aspect of the present disclosure wherein the angle of the incident light parallels that of the diffracted light.

FIG. 5 illustrates the use of a 2DPC sensor according to a preferred aspect of the present disclosure wherein incident light diffracts into a weaker back-diffracted and stronger forward-diffracted beams wherein the forward-diffracted bean is reflected by the mirror parallel to the back-diffracted beam toward the detector.

FIG. 6 shows a hydrogel layer according to a preferred aspect of the present disclosure comprising a molecular recognition agent and 2DPC self-assembling array tethered to Al or Ag mirror by a tethering hydrogel layer that does not constrain volume changes of the sensing hydrogel. The mirror reflects back the forward diffracted light. Whole blood may be analyzed preferably by introducing it from the bottom through the membrane filter layer. The membrane filter layer at the bottom preferably excludes blood cells.

FIG. 7a is a graph showing the L-phe concentration dependence of diffraction of imprinted IPC 3DPC in a pH=7.4 PBS buffer solution wherein the diffraction red shifted from 681 nm to 735 nm (total 54 nm).

FIG. 7b is a graph showing the dependence of imprinted IPN PCCA diffraction on concentration of L-phe, D-phe and L-trp wherein error bars measured for three replicate cycles of analyte addition.

FIG. 8 shows a fiber-optic reflection probe excited with white light and collects the 2DPC array diffracted light within a small range of wavelengths at the Littrow condition for CCD camera detection using the 2DPC array sensor according to a preferred aspect of the present disclosure.

FIG. 9a illustrates that in a sensor according to a preferred aspect of the present disclosure, monoclonal antibodies attached to 2DPC bind PSA, to swell the hydrogel.

FIG. 9b illustrates that in a sensor according to a preferred aspect of the present disclosure, different monoclonal antibodies bind PSA to create crosslinks to shrink the hydrogel.

FIG. 10 illustrates use of 2DPC pH sensor according to a preferred aspect of the present disclosure to measure proton secretion from epithelial cell monolayer in cell culture.

### DETAILED DESCRIPTION

It is to be understood that the descriptions of the present disclosure have been simplified to illustrate elements that are relevant for a clear understanding of the present disclosure, while eliminating, for purposes of clarity, other elements that may be well known. Those of ordinary skill in the art will recognize that other elements are desirable and/or required in order to implement the present disclosure. However, because such elements are well known in the art, and because they do not facilitate a better understanding of the present disclosure, a discussion of such elements is not provided herein. Additionally, it is to be understood that the present disclosure is not limited to the embodiments described above, but encompasses any and all embodiments within the scope of the description and the following claims. The terms % and wt. % have been used herein interchangeably to mean weight percent.

### DEFINITIONS

The following definitions apply to the terms as used throughout this specification, unless otherwise limited in specific instances.

As used herein, the tem′ “hydrogel” preferably, though not necessarily exclusively, refers to a network of polymer chains that are hydrophilic, sometimes found as a colloidal gel in which water is the dispersion medium. Hydrogels are highly absorbent (they can contain over 99.9% water) natural or synthetic polymers. Hydrogels also possess a degree of flexibility very similar to natural tissue, due to their significant water content.

As used herein, the term “photonic crystals” preferably, though not necessarily exclusively, refers to nanostructures composed of periodic dielectrics or metallo-dielectrics that affect the propagation of electromagnetic waves (EM) by defining allowed and forbidden electronic energy bands. Essentially, photonic crystals contain regularly repeating internal regions of high and low dielectric constant.

As used herein, the term “molecular recognition agents” preferably, though not necessarily exclusively, refers to the chemical or biological agent dispersed in the hydrogel layer that interacts with the analyte to be observed. The resultant interaction between molecular recognition agent and analyte results in either swelling or shrinking of a chemically sensitive hydrogel later.

As used herein, the term “crosslinks” preferably, though not necessarily exclusively, refers to bonds that link one polymer chain to another. They can be covalent bonds or ionic bonds. When polymer chains are linked together by cross-links, they lose some of their ability to move as individual polymer chains. For example, a liquid polymer (where the chains are freely flowing) can be turned into a “solid” or “gel” by cross-linking the chains together. In polymer chemistry, when a synthetic polymer is said to be “crosslinked”, it usually means that the entire bulk of the polymer has been exposed to the crosslinking method. The resulting modification of mechanical properties depends strongly on the crosslink density. Low crosslink densities decrease the viscosities of polymer melts. Intermediate crosslink densities transform gummy polymers into materials that have elastomeric properties and potentially high strengths. Very high crosslink densities can cause materials to become very rigid or glassy, such as phenol-formaldehyde materials.

As used herein, the phrase “bodily fluids” preferably, though not necessarily exclusively, refers to liquids originating from the bodies of living people. They include fluids that are excreted or secreted from the body as well as body water that normally is not such as, for example, amniotic fluid, aqueous humour and vitreous humour, bile, blood, blood serum, breast milk, cerebrospinal fluid, cerumen, chyle, endolymph and perilymph, feces, female ejaculate, gastric acid, gastric juice, mucus (including nasal drainage and phlegm), peritoneal fluid, pleural fluid, pus, saliva, sebum, semen, sweat, tears, vaginal secretion, vomit and urine.

As used herein, the term “monoclonal antibodies” (mAb or moAb) preferably, though not necessarily exclusively, refers to monospecific antibodies that are the same because they are made by identical immune cells that are all clones of a unique parent cell. Monoclonal antibodies have monovalent affinity, in that they bind to the same epitope. Given almost any substance, it is possible to produce monoclonal antibodies that specifically bind to that substance; they can then serve to detect or purify that substance.

As used herein, the term “CCD camera” preferably, though not necessarily exclusively, refers to a charge-couple device camera. A charge-couple device is a device for the movement of electrical charge, usually from within the device to an area where the charge can be manipulated, for example conversion into a digital value. This is achieved by “shifting” the signals between stages within the device one at a time. CCDs move charge between capacitive bins in the device, with the shift allowing for the transfer of charge between bins. CCD image sensors are widely used in professional, medical, and scientific applications where high-quality image data is required

### DESCRIPTION

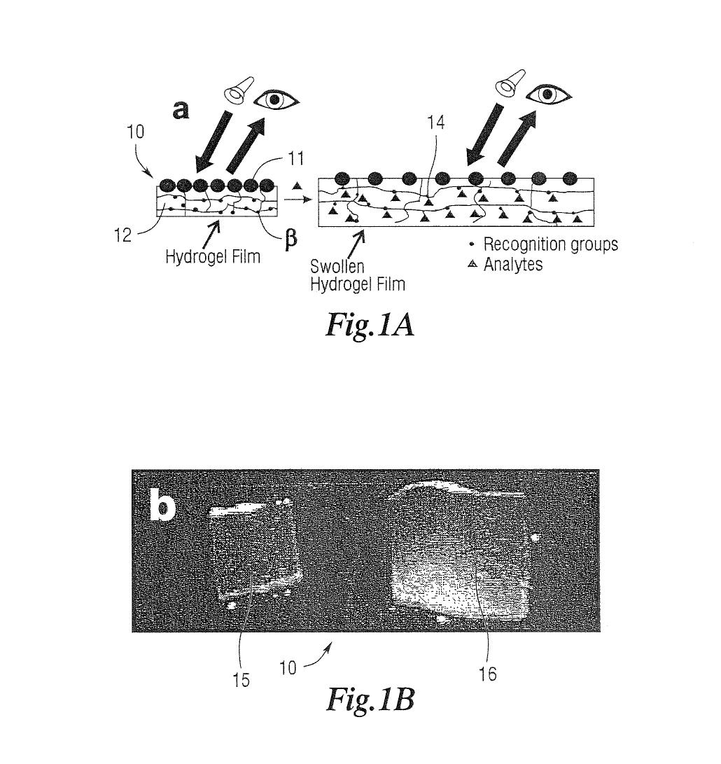

The present application describes nanoscale, ultrathin 2DPC that films can be chemically prepared to detect changes in concentrations of clinically important analytes as well as methods to easily self-assemble ultrathin, nanoscale 2D arrays of monodisperse particles whose interparticle array spacings change due to target analyte concentration changes. These 2DPC arrays brightly diffract incident light with extraordinary efficiencies. FIG. 1a illustrates a side-view of a preferred ultrathin 2DPC film 10 according to the present disclosure that has been prepared to detect a clinically important analyte. 2DPC film 10, comprises a 2DPC self-assembling array 11, a hydrogel layer 12 embedded with molecular recognition agent 13. The chemically sensitive hydrogels, that embed the 2DPC arrays, are tailored to undergo hydrogel volume phase transitions as the targeted analyte 14 concentration changes. The resulting spacing changes in 2DPC array 11 shift the wavelength of the intense 2DPC array diffraction. FIG. 1b illustrates a top view of a an ultrathin 2DPC film 10, before being exposed to an analyte 15 and after exposure 16. The diffracted wavelengths report with high sensitivity on the surrounding targeted analyte concentrations. The ultrathin 2DPC array in combination with a reflective surface diffracts more than 50% of the incident light making the diffraction color visually vivid. The concentrations of numerous analytes can be determined through the matching of the diffraction color to a color chart. Alternatively, the diffracted wavelength can be detected and the analyte concentration determined by using a simple cell phone camera based spectrometer, or by using a simple reflectance probe spectrophotometer.

One preferred aspect of the present disclosure involves novel chemical sensors that are useful in the diagnosis and treatment of disease. Often the diagnosis and treatment of disease requires determination of chemical species diagnostic of a patient's condition. Preferred chemical sensors of the present disclosure utilize novel nanoscale, two dimensional photonic crystals (2DPC) in a chemically-sensitive hydrogel. The preferred chemical sensors of the present disclosure provide point-of-care analyses and can be used in low resource environment situations.

Preferred chemically sensitive hydrogels, comprising one or more molecular recognition agents and a 2DPC self-assembling array of the present disclosure are useful as new sensing materials to determine low concentration of analytes in ultrasmall sample volumes. These sensors utilize hydrogel volume transitions to PC diffraction readouts in a manner reminiscent to that described for various 3DPC array sensors. Holth, J H; Asher, S A; Intelligent Polymerized Crystalline Colloidal Array Hydrogel Film Chemical Sensing Materials, Nature 1997, 389, 829-832. However, the 2D nature of the material chemical sensing response and the 2DPC diffraction phenomena uniquely and surprisingly enables the sensing of macromolecular analytes. Further, the 2DPC hydrogel arrays surprisingly permit much higher analyte sensitivities. The brighter 2DPC diffraction results in very intense optical readouts that are easy to visually observe. The uniqueness of the phenomenon can be understood by comparing the 2DPC diffraction intensity to fluorescence sensing. Essentially light wavelengths in the range meeting the 2DPC Bragg condition incident on the 2DPC array diffract into a spectrum where each wavelength diffracts into a narrow angle of about 0.01 str (>10% of the intensity, for argument, 50-80% actually). In contrast, a 1 cm² are 10 μm thick solution with a 10⁻⁶ M concentration fluorescent dye with a about 10⁻¹⁶ cm² absorption cross section and 100% quantum yields shows an absorption of 0.01% of the incident light and only 10⁻⁵% of the fluorescent light is emitted into the same small 0.01 str angular width.

The 2DPC diffracted wavelength is a sensitive function of the 2DPC hydrogel volume which is determined by the derivative of the change in the Gibbs' free energy (ΔF_{T}) with the change in volume: ∂ΔF_{T}/∂V=∂/∂V[ΔF_{M}+ΔF_{I}+ΔF_{E}]=Π_{M}+Π_{I}+Π_{E}=0. Xu, X; Goponenko, A V; Asher, S A; Polymerized PolyHEMA Photonic Crystals pH and Ethanol Sensor Materials, J. Am. Chem. Soc., 2008, 130, 3113-3119. This relationship indicates that for any change in the sensor chemical environment, the hydrogel volume will evolve, until the volume-derivative of the hydrogel's total Gibb's free energy equals zero. The volume-derivative of ΔF_{T} is the total osmotic pressure, Π_{T}. Changes in the total free energy of a hydrogel results from changes in the free energies associated with the free energy of mixing, ΔF_{M}, the free energy associated with electrostatic interactions, ΔF_{T}, and the elastic free energies contributed by crosslinks in the hydrogel, ΔF_{E}. The system will evolve until the total osmotic pressure is zero, which occurs only when the osmotic pressure associated with mixing, Π_{M}, the electrostatic osmotic pressure, Π_{I}, and the elastic restoring force osmotic pressure, Π_{E}, counterbalance one another.

Changes in the free energy of mixing, ΔF_{M}, derive from changes in the chemical nature of the hydrogel surfaces exposed to water. Subtle changes in chemical structure give rise to large hydrogel volume changes. Larger changes in ΔF_{M} occur due to ionization. Binding of proteins, that change the hydrogel surface hydrophilicity and hydrophobicity, should have larger impacts on ΔF_{M} and on the resulting hydrogel surface volume.

Changes in the ionic state of the hydrogel cause changes in both the free energy of mixing and the electrostatic free energies. The electrostatic effects can be very large in low ionic strength solutions, but they generally are smaller for high ionic strength samples such as bodily fluids. Often it is possible to reduce the sample solution ionic strength to take advantage of these large ionic free energy changes, ΔF_{I}.

Changes in the elastic free energies, ΔF_{E}, also result from changes in the crosslink density that occurs due to making or breaking crosslinks. These crosslinks can derive from covalent bond formation or association phenomena, such as hydrophobic cluster formation or hydrogen bonding. These elastic free energies have an entropic origin, because they derive from constraints in the possible conformations of the hydrogel chains as the volume changes.

Thus, subtle chemical differences can result in large hydrogel volume changes, which result in large 2DPC array diffraction changes. The diffraction wavelength for backscattering diffraction in the Littrow configuration will depend upon the linear dimensions of the hydrogel. For first order 2DPC array diffraction: λ₀=2 d sin θ, where λ₀ is the wavelength of light in vacuum, d is the 2DPC array lattice spacing that is diffracting and θ is the common angle of the incident and diffracted light beams relative to the 2DPC array normal.

Preferred 2DPC array chemical sensors of the present disclosure can use any chemical phenomenon that actuates one or more free energy changes discussed above. Effectively, these changes in free energies directly translate into changes in the osmotic pressure within the hydrogel. The volume continues to change until the net osmotic pressure equals zero. This is identical to the statement that the chemical potential of water within the hydrogel must be identical to that in the exterior analyte solution.

3D arrays for sensing the changes in the free energy of mixing, such as the creatinine sensor that titrates a pendant phenol group, for example or that actuate crosslinking changes such as the glucose sensor and the metal cation sensors have been previously demonstrated. Further, ionic free energy based sensors such as the pH sensors that titrate carboxyl residues, and crown ether based Pb²⁺ sensors have been demonstrated.

Preferred 2DPC array sensors of the present disclosure utilize an about 500 nm thick 2D hexagonal array of nanoscale periodic particles polymerized into a chemically sensitive hydrogel. This array diffracts light in the visible (or UV or IR) spectral region due to the spacing of the 2D periodic array of colloidal particles. This material also contains covalently attached molecular recognition agents or contains imprinted sub nano-binding domains that bind the analytes of interest. Interaction of the analyte with the 2DPC array spacing shifts diffraction wavelength and the visually observed color. The diffracted wavelength change reports on the identity and concentration of the analyte of interest. The 2DPC array sensors of the present disclosure offer novel, inexpensive, and practical technology for fabricating clinical sensors for use in clinical chemistry, for biological investigations and for use in fabricating extraordinarily simple, inexpensive point-of-care clinical sensors.

Preferably, the 2DPC array chemical sensors of the present disclosure are ultrathin and a major part of the response derives from chemical interactions at the surface of such sensors. This, thus, enables the use of the chemically sensitive hydrogel layers, comprising one or more molecular recognition agents and a 2DPC self-assembling array of the present disclosure for detection of macromolecular species. Examples would include using attached antibodies to identify and determine protein concentrations including cancer marker proteins, as well as other markers that are used for disease diagnosis and analysis platforms. Typically, a broad range of hormones, such as thyroid hormones, steroids and glycoproteins, are analyzed on automated immuno-analyzers. Cancer markers, such as prostate-specific antigen (PSA), are recognized as approved diagnostic tests and also quantitated on automated immuno-analyzers. Preferred 2DPC array chemical sensors of the present disclosure can be used to determine the existence, in samples, of particular DNA sequences by attaching single strand DNA of a particular sequence to the 2DPC array hydrogel. Any DNA in solution that hybridizes will increase the attached charge and cause the 2DPC lattice spacing to dramatically increase, thereby dramatically shifting the diffraction wavelength.

Prostate specific antigen (PSA) was discovered roughly 30 years ago, and is the most widely utilized tumor marker. PSA, which is approved for cancer screening in combination with a digital rectal exam, is a serine protease with chymotrypsin-like activity. PSA acts on specific antigens in the rodent seminal plasma clot after ejaculation resulting in liquefaction. PSA also acts on specific proteins in seminal plasma to maintain the liquid consistency. Most of the PSA found in seminal plasma occurs in its 30 KD form. This “free” fraction contains both enzymatically active and inactive forms. Two complexed forms of PSA are found in serum along with the free fraction. One form is a complex of PSA and α₁-antichymotrypsin of molecular weight ˜100 KD. This complex is usually about 80% of the serum immunoreactive PSA.

The PSA molecule contains six antigenic sites. One is masked following binding to α₁-antichymotrypsin. Measurements of total PSA can utilize any of the remaining antigenic sites. Most current assays are of the “sandwich” type, based on the use of one polyclonal and one monoclonal antibody, or on the use of two monoclonal antibodies. While early kits from varying manufacturers yielded differing serum values, recent assays from different manufacturers now recognize similar epitopes on PSA and yield similar results.

Current assay limits are in the range of 0.01-0.1 μg/L. While PSA has been a significant aid to the early diagnosis of prostate cancer, of 8-15% of men over the age of 50 with PSA levels >4 μg/L, most will have either benign prostatic hypertrophy or prostatitis. This has confounded the interpretation of the test. Moreover, 5-10% of patients with PSA between 2-4% μg/L may still have prostate cancer. Despite these limitations, PSA screening and the use of PSA to follow reoccurrence of prostate cancer is now standard. Because of the widespread use of PSA in the early screening for cancer, PSA is an ideal target for an antibody-antigen 2DPC array sensor according to the present disclosure. Preferred PSA chemical sensors of the present disclosure may be fabricated by attaching antibodies specific for PSA. Preferably, avidin-biotin coupling is used to attach the antibodies. Biotin acrylate monomers were copolymerized within the 3DPC to bind avidinated proteins. Preferably the antibodies are avidinated using standard chemistry. These antibodies will then bind to the biotinylated 2DPC. The avidinated antibodies may then be copolymerized with biotin acrylate monomers and the 2DPC self-assembling arrays to form the chemically sensitive hydrogel layers. Additionally, the antibodies can be attached to a previously formed hydrogel 2DPC self-assembling array layer by the use of glycidyl methacrylate monomers. For this approach the antibody is chemically attached to the glycidyl group. The antibody can be attached to a previously formed hydrogel 2DPC self-assembling array layer by EDC chemistry. For this approach the hydrogel is preferably prepared with carboxylates to which the amines of the antibodies are attached by the EDC reaction.

The 2DPC surface bound antibodies will bind the PSA antigen. This will alter the free energy of mixing. Since the outside surfaces of proteins are generally hydrophilic, the increased protein surface area will increase the free energy of mixing of the sensing hydrogel. This will increase the hydrogel volume and redshift the diffraction wavelength. Binding two different antibodies which are specific to different PSA antigenic sites may also be possible in which case antibody binding will form crosslinks on the 2DPC surface.

Preferably, antibodies with the required high association constants for efficient binding of PSA to the 2DPC bound antibodies are employed. Purified free PSA (fPSA), PSA complexed to α₁-antichymotrypsin (PSA-ACT) and crosslinks to shrink the hydrogel, monoclonal antibodies that recognize both free and complexed PSA are available from Fitzgerald Industries International. These antibodies show affinity constants of 10⁸-10¹⁰ L·mol⁻¹. Antibodies to PSA1 (ab403) are available from Abcam plc., with an affinity constant of ˜10¹¹ L·mol⁻¹.

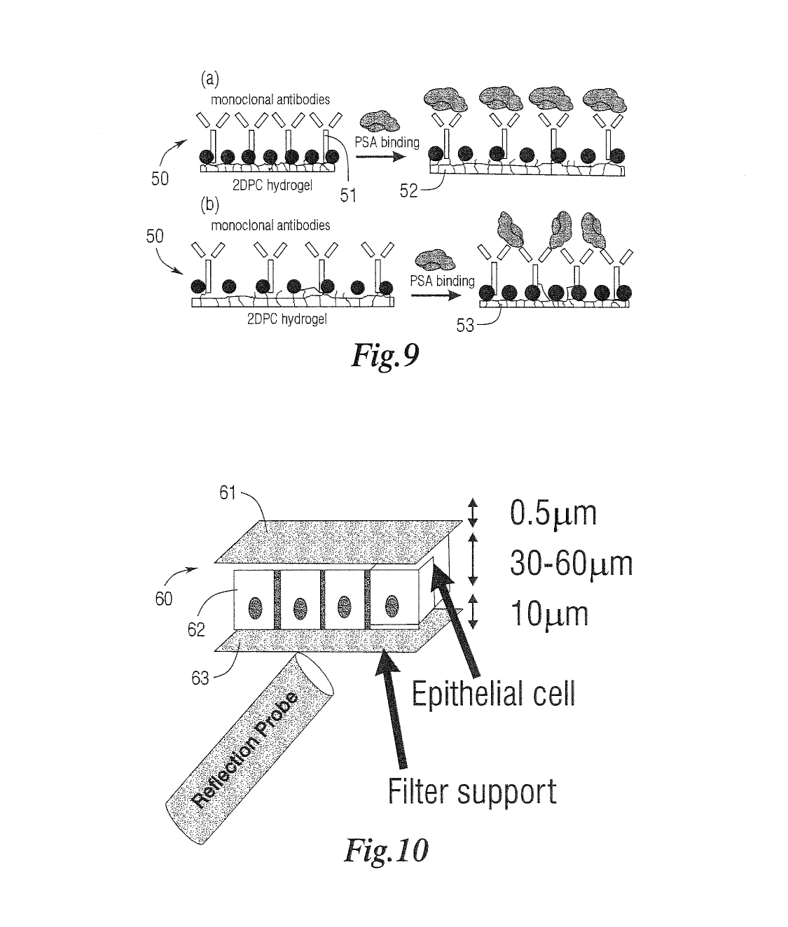

FIG. 9 illustrates a preferred chemical sensor of the present disclosure configured to detect the presence and/or concentration of PSA 50. The PSA sensor 50 may be configured such that the monoclonal antibodies 51 individually bind the PSA and cause the hydrogel to swell as at 52 or they may be configure such the monoclonal antibodies crosslink to bind the PSA and cause the hydrogel to shrink as at 53.

Many of the preferred chemically sensitive hydrogels of the present disclosure contain specific molecular recognition agents tailored to determine a specific analyte. Additional aspects of the preferred sensors of the present disclosure anticipate the use of analyte imprinting in the chemically sensitive hydrogel as was described by Hu et al. Hu, X B; An, Q; Li, G T; Tao, S Y; Liu, B; Imprinted Photonic Polymers for Chiral Recognition. Angewandte Chemie-International Edition, 2006, 45(48), 8145-8148. An acrylamide hydrogel crosslinked with bisacrylamide will be infiltrated with acrylic acid and in the presence of the targeted analyte L-phe. UV photopolymerization will generate an interpenetrating network (IPN) imprinted for L-phe. After washing out the L-phe the sensor will be highly imprinted for L-phe.

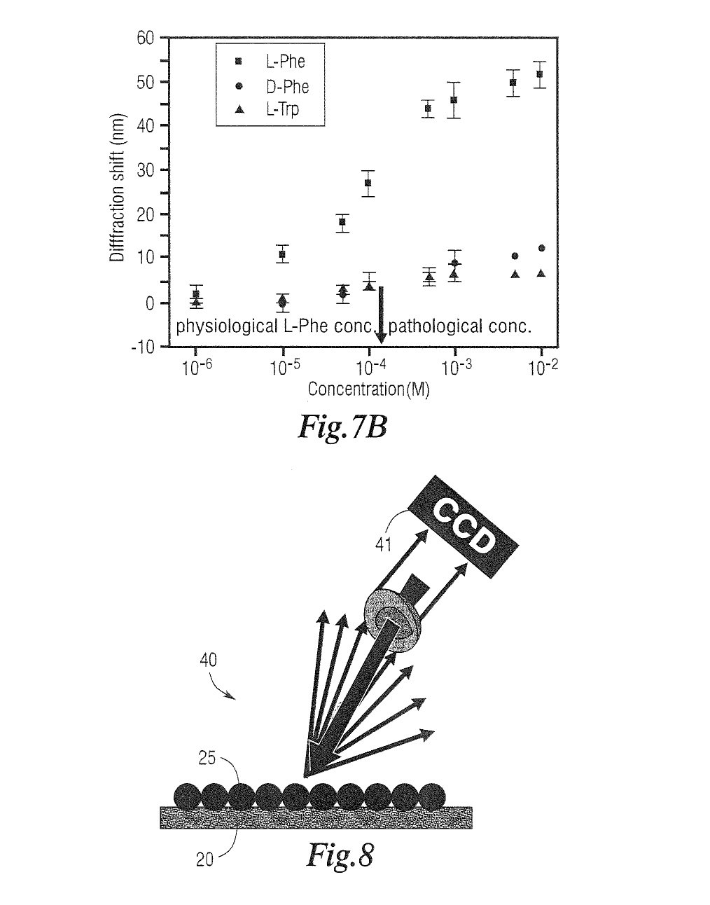

3DPC from polystyrene spheres were formed within an acrylamide hydrogel crosslinked with bisacrylamide. The 3DPC was infiltrated with acrylic acid in the presence of the targeted analyte L-phe. UV photopolymerization generated an interpenetrating network (IPN) imprinted for L-phe. After washing out L-phe the 3DPC sensor was highly sensitive to the L-phe concentration (FIG. 7). The sensor was selective for L-phe as evident from lack of impact on the diffraction by D-phe or other amino acids. Preferably, imprinting will incorporate interpenetrating hydrogels in the 2DPC containing the necessary functional groups that can form nanocavities with stabilizing intermolecular interactions in the presence of the analyte.

FIG. 7a illustrates L-phe concentration dependence of diffraction of imprinted IPN 3DPC in a pH=7.4 PBS buffer solution. The diffraction red shifted from 681 nm to 735 nm (total 54 nm). FIG. 7b illustrates dependence of imprinted IPN PCCA diffraction on concentration of L-phe, D-phe, and L-trp. Error bars measured for three replicate cycles of analyte addition.

Blood osmolality is an important diagnostic reflecting a patient's fluid balance and is typically determined by freezing point depression and typically requires a dedicated clinical instrument. Preferred 2DPC array sensors of the present disclosure can be use to fabricate a simple osmolality sensor that operates on the basis of the transient water chemical potential difference between the interior of 2DPC array hydrogel layer containing pure water compared to the added blood sample.

A transient osmotic pressure difference will occur between the 2DPC array hydrogel interior and the blood sample due to the high concentration of dissolved species in blood. A preferred osmolality sensor according to the present disclosure is made utilizing a nonfunctionalized 2DPC hydrogel containing a polystyrene 2DPC array embedded in an acrylamide hydrogel.

Initially, such 2DPC array osmolality sensor according to the present disclosure is calibrated against simple solutions of water containing species such as salts, sugars, proteins, etc. The blueshift diffraction response is then calibrated as water flows out of the 2DPC hydrogel into the high osmolality solution due to the transient osmotic pressure difference that tries to establish identical chemical potentials for the water. Water will dominate the transient response since it is the fastest diffusing species. This transient response has already been observed in studies of prior 3DPC creatinine sensor response to serum samples. It is expected that the magnitude of the blue shift will directly correlate with the osmolality of blood (after about two minutes or less following blood addition).

Preferably, 2DPC array sensor thickness and the crosslink density are designed to obtain the necessary sensitivity and temporal response required for the osmolality determinations (260-320 mMoles/kg). Lacking a sufficient understanding of the hydrogel polymer self diffusion constant to accurately predict the ideal thickness, the photonic crystal (PC) composition and thickness are preferably adjusted to give an optimum measurement interval. Although 2DPC array osmolality sensors according to the present disclosure are expected to measure osmolality from fingerstick blood samples, initial development studies will compare the osmolality response to that of the clinical lab for heparanized discarded blood samples and also compare osmolality measurements between heparanized and nonheparanized blood.

A transient osmotic pressure difference will occur between the 2DPC array hydrogel interior and the blood sample due to the high concentration of dissolved species in the blood. A 2DPC array osmolality sensor according to the present disclosure preferably is calibrated against simple solutions of water containing species such as salts, sugars, proteins, etc. These solutions will allow calibration of the blueshift diffraction response as water flows out of the 2DPC hydrogel in the high osmolality solution due to the transient osmotic pressure difference that tries to establish identical chemical potentials for the water. Asher, S A; Sharma, A C; Goponenko, A V, Ward, M M; Photonic Crystal Aqueous Metal Cation Sensing Materials. Anal. Chem., 2003, 75, 1676.

Preferred chemical sensors of the present disclosure useful as blood pH sensors have a hydrogel layer preferably utilizing acrylic acid so as to incorporate carboxyl groups. As the pH increases, the 2DPC array diffraction shifts due to carboxyl group titration.

However, a blood pH sensor will not be able to rely on the electrostatically actuated Donnan potential. Blood pH sensors according to the present disclosure preferably are based on a nitrophenol titration model. Nitrophenol deprotonation to the nitrophenolate form will increase the free energy of mixing and increase the 2DPC lattice constant. Preferably, blood pH sensors of the present disclosure are optimized in order to be able to measure <0.05 pH unit differences over the pH range 6.9 to 7.6. Preferably, the response may be adjusted by varying the nitrophenol loading and by controlling the crosslink density.

2DPC array chemical sensors of the present disclosure preferably are useful for the detection and quantitation of several biologically important cationic species. 2DPC array sensors according to the present disclosure for sodium, potassium, calcium, magnesium and ammonium preferably are prepared by covalently attaching ion selective chelating agents onto the hydrogel. Numerous molecules are known to bind these molecules such as crown ethers, calixarenes, etc. The appropriate chelating agent may be prepared with vinyl groups and copolymerized with the 2DPC array hydrogel. Additionally, Ca²⁺ binding proteins such as Calmodulin preferably may be used for highly selective binding of Ca²⁺.

Preferably, actuation of the hydrogel volume changes in high ionic strength solutions are accomplished utilizing changes in the free energy of mixing of the molecular recognition groups on ion binding. Binding of a cation to a crown ether, for example, will give a more favorable free energy of mixing, that will redshift the diffraction. Ion binding will impact the free energy of mixing similarly to that occurring upon nitrophenol deprotonation, assuming that electrostatics and solvent polarization around the ion dominate the free energy of mixing. For clinical applications, sensors according to the present disclosure for relatively high concentration species such as Na⁺ (˜150 mM) and K⁺ (4 mM) are preferred utilizing high concentrations of chelating agents that have modest analyte affinities. Since the incorporation of greater than 0.03 M 4-acryloylaminobenzo-18-crown-6 into 3D Pb²⁺ sensors is easily accomplished. Recognition agents sufficiently selective such that there is no confounding between ions as different as Na⁺ and K⁺ are also expected. Preferably, for these analytes, simple crown ethers with appropriately different interior ring diameters may be utilized. While high concentration analytes will likely require moderately crosslinked 2D sensing hydrogels.

More overlap in affinities between ions, such as Ca²⁺, Mg²⁺ and Zn²⁺, will require the use of multiple sensors with different ligand affinities. By knowing the selectivity coefficients the diffraction response can be monitored, and the concentrations of different species can be determined by linearly solving the set of appropriate equations. Preferably, the range of response and the sensitivity of the photonic crystal sensors are controlled by adjusting the molecular recognition agent concentration, its analyte affinity, and/or the hydrogel crosslink density.

There are numerous groups around the world developing selective chelating agents to bind different species in solution, such as the IBC Company, a leader in the synthesis of chelation compounds for the selective uptake of metals and other aqueous species. Also, commercially available crown ethers are preferred for working for Na⁺ and K⁺, while commercially available selective binding proteins may be ideal for Ca²⁺ and Mg²⁺. Macrocyclic compounds, such as, 15-crown-5 ether groups have been shown to bind NH₄⁺ ions due to a favorable arrangement of oxygen donors in the ring. In addition, sensors for ammonia have utilized Dickert et. al's, approach where, nickel (II) chloride surrounded by azacrown ethers creates a system in which ammonia replaces the interaction between the nickel and chloride, creating an ion which is detected by conductivity measurements at ammonia concentrations as low as 500 ppm. Hughes et. al. developed triarylborane complexes which bind ammonia. The ammonia nitrogen donates electrons to the electron deficient borane, while the ammonia hydrogens are stabilized by ester oxygens on the aryl groups. Further, IBC Company has synthesized a new chelating complex for NH₄⁺ that is preferred for fabricating a photonic crystal ammonia sensor. Preferably, all of the sensors above can be used for trace detection if nonbound ions contributing to the high ionic strength are quickly washed away, as demonstrated in trace 3D Pb²⁺ studies. In this case, it is easy to determine ppb and ppm concentrations of these analytes; the sensors transiently swell after the unbound ions are washed out and the bound ions remain due to their slower off-rates.

Preferred chemical sensors of the present disclosure are useful for the detection and quantitation of several biologically important anionic species. Sensors for chloride, phosphate and bicarbonate may be prepared by using the rotaxane ligands of Hancock or the ligands taught by Hisamoto for a chloride sensor. Beer, P D; Hancock, L M; Gilday, L C, Carvalho, S; Costa, P J; Felix, V; Serpell, C J; Kilah, N J; Rotaxanes Capable of Recognizing Chloride in Aqueous Media, Chem. Eur. J., 2010, 16, 13082-13094. and Suzuki, K; Hisamoto, H; Miyshita, N; Watanabe, K; Nakagawa, E; Yamamoto, N; Ion Sensing Film Optodes: Disposable Ion Sensing of Na⁺, K⁺, Ca2⁺ and Cl⁻ Concentrations in Serum, Sensors and Actuators B, 1995, 29, 378-385. A complexing agent taught by Hennrich or the selective ligand developed by Yan and Li will be used for the bicarbonate sensor. Hennrich, G; Sonnenschein, H; Resch-Genger, U; Fluorescent Anion Receptors with Iminoylthiourea Binding Sites-Selective Hydrogen Bond Mediated Recognition of CO₃²⁻, HCO₃⁻ and HPO₄²⁻, Tetrahedron Lett., 2001, 42, 2805-2808. and Yan, H J; Li, H B; Urea Type of Fluorescent Organic Nanoparticles with High Specificity for HCO₃⁻ Anions, Sensors and Actuators B-Chemical, 2010, 148(1), 81-86. Either the Zn(II) ligand of Han and Kim or the Cu(II) of Saeed will be used to prepare the phosphate sensor. Han, M S; Kim, D H; Naked-Eye Detection of Phosphate Ions in Water at Physiological pH: A Remarkably Selective and Easy-to-Assemble Colorimetric Phosphate-Sensing Probe, Angewandte Chemie-International Edition, 2002, 41(20), 3809-3811 and Saeed, M A; Powell, D R; Hossain, M A; Fluorescent Detection of Phosphate Anion by a Highly Selective Chemosensor in Water, Tetrahedron Lett., 2010, 51(37), 4904-4907.

Although there is a very extensive literature on aqueous solution cation chelation compounds, there are significantly fewer anion chelation compounds reported. In previous work, use of the selective Cl⁻ ligands pioneered by Prof. Bowman-James's group was attempted, but it was determined that the Cl⁻ affinities in mainly aqueous solutions were too small. Presently, the use the rotaxane ligands that Hancock et. al. showed selectively bound Cl⁻ in mixed organic/aqueous solvents are preferred. Their dicationic [2]rotaxane 10 (PF₆)₂ ligand binds Cl⁻ with association constants of 3000 M⁻¹ and 500 M⁻¹ in 10% water and 35% water, respectively. If necessary, we will use the ligands Hisamoto et al utilized to fabricate an optrode to determine Cl⁻ in serum. If necessary, the hydrophobicity of preferred 2DPC hydrogels can be increased to increase the Cl⁻ ligand affinities.

HCO₃⁻, the third most abundant extra-cellular ion in plasma, next to Na⁺ and Cl⁻, is routinely used to classify the acid-base status of ill patients. Serum HCO₃⁻ is generally measured via enzymatic analysis or ionselective electrode analysis. This measurement requires minimal intervals between sample collection and analysis. Because of the importance of HCO₃⁻ in acid-base physiology, preferred sensors for HCO₃⁻ preferably utilize a complexing agent similar to that of Hennrich et. al., or a recent selective ligand developed by Yan and Li. Also, because phosphate is very important physiologically, preferred sensors to determine the dominant physiologically forms HPO₄²⁻ and H₂PO₄⁻ preferably utilize the high affinity Zn(II) ligand of Han and Kim or the Cu(II) ligand of Saeed, et al.

Preferred 2DPC array chemical sensors of the present disclosure are useful for the detection and quantitation of toxic metals such as mercury. A preferred 2DPC array chemical sensor for toxic metals can be prepared from the dianionic ligand 1,3-benzenediamidoethanethiolate that irreversibly binds Hg²⁺ to form a remarkably stable complex that is unaffected by oxidizing agents or pH. Preferably, this ligand will be synthesized with added functionalities to make it easy to attach to the hydrogel. The dianionic ligand charge will be neutralized upon Hg²⁺ binding. This will actuate a 2DPC array shrinkage due to the decreased Donnan potential and free energy of mixing. Alternately, other complexing species such as dithia-substituted 18-crown-6 ethers that bind Hg²⁺ with enormously high stability constants of ˜10²⁵ can be used. The increased ligand charge attached to the 2DPC will result in an increased Donnan potential, as well as, an increased free energy of mixing. The magnitude of the redshift will be directly related to the original Hg²⁺ concentration. These sensors may require more blood than can be obtained by just a fingerstick.

Urea, the chief end product in protein catabolism in humans, is produced in the liver and is normally excreted in urine by the kidney. The Blood Urea Nitrogen (BUN) level is a vital indicator of protein metabolism and is used to identify liver and kidney disorders. For healthy adults, the reference interval for plasma or serum urea is 6 to 20 mg/dL (2.1-7.1 mM). There have been many different methods developed for determining urea, most of which utilize the enzyme urease, an enzyme found in bacteria, yeast, and higher plants. The main source for urease is Jack Bean meal (Canavlia ensiformis), from which it is crystallized and studied. Urease specifically catalyzes the hydrolysis of urea to ammonia and carbonic acid.

At physiological pH, the reaction of urea with urease is:

(NH₂)₂CO+3H₂O→2NH₄⁺+OH⁻+HCO₃⁻

Given that the bicarbonate ion has a pKₐ=10.33, the initially neutral pH will increase due to the formation of OH⁻. A 2DPC sensor of the present disclosure will preferably use a similar approach that was used to develop the 3D creatinine sensor. Urease and 2-nitrophenol will be covalently attached to the 2DPC hydrogel. As urease hydrolyzes urea, the pH increase will deprotonate the phenol, swell the hydrogel and shift the diffraction. In support of this approach it is noted that Zeng et al used our approach to trap urease within a 3DPC hydrogel in order to sense urea in low ionic strength solution. Their 3DPC sensor redshifted its diffraction due to the pH increase that deprotonated the hydrogel carboxyl groups. Preferred 2DPC sensors of the present disclosure will sense urea in the high ionic strength pH 7.4 environment of blood.

Uric acid, the major product of protein catabolism, is a major nitrogenous component in urine. Uric acid is normally present in blood. The assay of uric acid in bodily fluids is clinically very important. Hyperuricemia is associated with a number of conditions, such as leukemia, gout diabetes, high blood pressure, high cholesterol, kidney disease, and heart disease. Hyperuricemia is defined by serum or plasma uric acid concentrations greater than 7.0 mg/dL (0.42 mM) in men and 6.0 mg/dL (0.36 mM) in women. The standard analysis for uric acid is with uricase, which catalyzes the uric acid into allantoin and producing hydrogen peroxide. The chemical sensors of the present disclosure will incorporate uricase to catalyze the uric acid. The resulting hydrogen peroxide will oxidize pendent sulfhydryls in the hydrogel layer to dissulfide crosslinks and thereby cause the hydrogel to shrink and shift the diffraction.

Numerous methods have been developed for the determination of uric acid, most of which utilize the enzyme uricase. Uricase is a purine enzyme whose main sources include Aspergillus flavus, Candida utilis (˜73 KD), Bacillus fastidiosis, and hog liver. Uricase selectively catalyzes the oxidation of uric acid to allantoin, producing hydrogen peroxide:

uric acid+2H₂O+O₂→allantoin+H₂O₂+CO₂

A 2DPC array sensor of the present disclosure for uric acid preferably will utilize the H₂O₂ generated to oxidize pendent sulfhydryls to disulfide crosslinks in the 2DPC hydrogel. Preferably, uricase will be attached to the surface of the 2DPC array hydrogel and incorporate free sulfliydryl groups within and on the hydrogel. These sulfliydryls will be oxidized to form disulfide bonds to shrink the hydrogel. Hydrogels with both pendant sulfhydryls and disulfide crosslinks that clearly demonstrate diffraction shifts upon formation and breakage of crosslinks have been synthesized. Molecular oxygen can also oxidize sulfhydryls to disulfides, but these reactions are slower, and can additionally be impeded by removing oxygen.

The development of a sensing motif that uses H₂O₂ to monitor enzyme turnover, will enable numerous other sensors, such as a lactic acid sensor, for example. Preferably, the reaction catalyzed by lactate oxidase will be utilized:

Lactate+O₂pyruvate+H₂O₂

2DPC array sensors of the present disclosure for monitoring cell cultures and tissue surfaces to visualize and sense excreted analytes could aid many biological investigations. The diffraction color of a small area 2DPC array sensor targeted to an important analyte situated on top of a cell or tissue surface will accurately report on analyte flux. 2DPC array sensors of the present disclosure preferably may be used to dynamically measure cell culture surface pH and for examining the response of these cells to drugs that alter proton transport. This will alleviate the present difficulties in studying the chemical environmental dependence of proton transport by membrane transport proteins in cells and tissues; existing methods to determine extracellular pH are cumbersome. For example, pH sensitive fluorescent dye methods often utilize intense UV light in confocal microspectroscopy measurements. The illumination and the dyes can stress the cells or tissues studied. The alternative microelectrochemical methods are not readily commercially available; their use requires highly trained personnel to fabricate and carry out the measurements.

The 2DPC pH sensors of the present disclosure are also preferably useful to develop novel measurements to augment Dr. Pastor-Soler's acid-base homeostasis studies that examine regulation of kidney and male reproductive tract pH by the vacuolar H⁺-secreting ATPase (V-ATPase). V-ATPase, that uses ATP hydrolysis to pump protons across membranes, maintains the stable low pH of the male reproductive tract lumen required to promote sperm maturation. V-ATPase also controls kidney excretion of the daily non-volatile acid load. Dysfunction of V-ATPase leads to renal tubular acidosis, a disease with severe systemic sequelae. The study of the regulation of the V-ATPase and other proton transport proteins has been challenging due to the existing experimental challenges of pH microsensing. The 2DPC pH sensors of the present disclosure should help increase the understanding of regulation of the V-ATPase proton transport in cell cultures derived from kidney epithelium and in the male reproductive tract.

The 2DPC pH sensors of the present disclosure are also useful to measure extra-cellular pH of polarized apical proton-secreting cells to examine the V-ATPase response to agonists/antagonists or to knock-down/overexpression of V-ATPase or potential regulatory proteins. The Clone C kidney cell line that expresses high levels of VATPase at their apical membranes (FIG. 10) will be used and the 2DPC pH sensors of the present disclosure preferably are placed on cell monolayers grown on 10 μm-thick transparent Transwell supports (˜33 mm²)

Initially ˜10 mm² sensors are preferred that can easily be situated and monitored. While later, much smaller sensors (possibly ˜10 μm², smaller than the cell apical membrane area) will be employed. The small 2DPC sensors preferably will utilize magnetic particles pioneered earlier so their placement can be controlled with small magnets. Diffraction preferably is monitored by exciting in the Littrow configuration by using a reflection probe to sense the weaker, but sufficiently intense backdiffracted light.

Previously it has been found that protein kinase A (PKA) increases V-ATPase concentrations at the apical membrane. The 2DPC pH sensors of the present disclosure allow monitoring of Clone C cell surface pH changes before and after exposure to a PKA agonist such as a cAMP analog. To verify that the pH changes result from V-ATPase V-ATPase activity is preferably inhibited with bafilomycin. Previous measurements saw changes of 0.005-0.020 pH units/min in 1 mL bathing buffer solutions. Higher pH changes are expected at the cell culture surface.

Additional applications of the 2DPC array sensors of the present disclosure include sensing the pH of ex vivo cut-open vas deferens preparations from anesthetized rats. For example, such 2DPC sensors preferably may be used to measure extracellular pH changes in real time in response to drugs known to activate/inhibit V-ATPase. In addition, such 2DPC sensors preferably may be used to study the effects of knockdown or overexpression of regulatory proteins that may affect V-ATPase function by using standard molecular biology approaches. Such studies will give deep insights into drug regulation of proton transport in functioning tissue.

The 2DPC array chemical sensors of the present disclosure have application for determining cell culture surface and tissue surface analytes. The chemical sensors of the present disclosure can be used to dynamically measure cell culture surface pH and to examine the response of these cells to drugs that alter proton transport. The chemical sensors of the present disclosure preferably measure the pH of the cells to be studied in a non-cumbersome manner that is also non-stressful to the cells/tissues being studied. FIG. 10 illustrates a cell surface/tissue surface sensor 60. Sensor 60, comprises a 2DPC monolayer 61 placed directly on cell monolayers 62 that are grown on 10 μm-thick transparent Transwell supports 63.

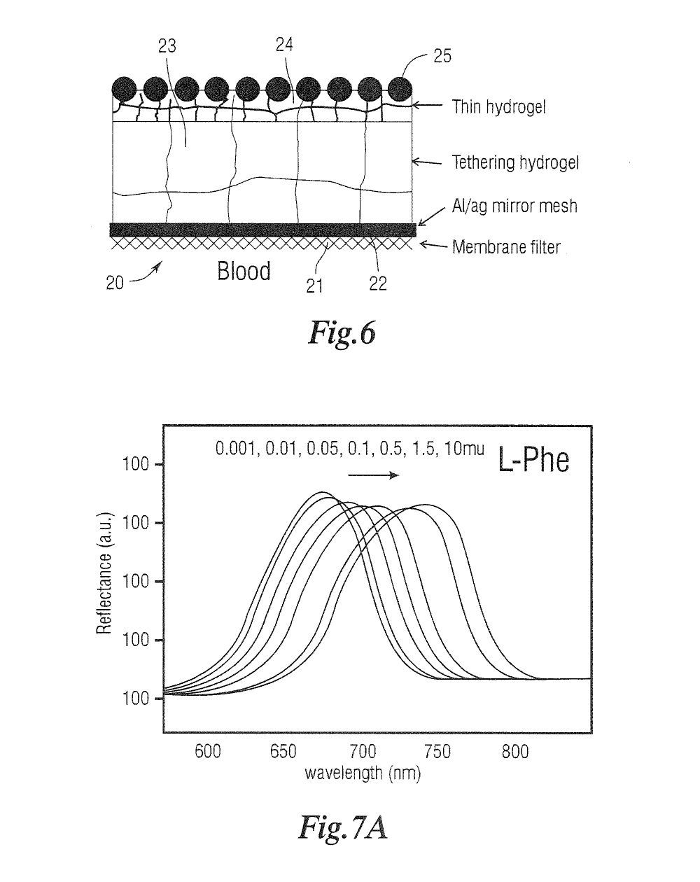

FIG. 6 illustrates a typical two-dimensional cross section of a chemical sensor 20 of the present disclosure. Chemical sensor 20, comprises a membrane filter layer 21, a mirror layer 22, a tethering hydrogel layer 23 and a hydrogel layer 24 having a 2DPC array 25 therein.

The chemical sensors 20 are assembled as a series of layers with the first, or bottom, layer comprising a membrane filter layer 21. The membrane filter layer 21 chosen for the chemical sensors 20 of the present disclosure preferably are liquid permeable with a porosity such that the analyte to be observed will readily pass through the layer 21. Thus, when the sample or bodily fluid to be analyzed is blood, the membrane filter layer 21 will be designed such that the porosity will filter out the blood cells while allowing the blood plasma to readily pass through the layer 21.

Directly bonded to the membrane filter layer 21 is a mirror layer 22. The mirror layer 22 chosen for the chemical sensors 20 of the present disclosure preferably is liquid permeable with a porosity such that the analyte to be observed will readily pass through the layer 22. The mirror layer 22 may be fashioned from any suitably reflective material, such as, for example, aluminum and/or silver.

Preferably, the mirror layer 22 is prepared before addition of the tethering hydrogel layer 23. The mirror layer 22 may be prepared in any way to one skilled in the art of polymer science such as, for example, the addition of vinyl groups to its surface.

Directly bonded to the mirror layer 22 is the tethering hydrogel layer 23. The tethering hydrogel layer 23 is attached to the prepared mirror layer 22 as a relatively rigid polymer sheet containing a high density of sacrificial, easily broken crosslinks and a lower density of chemically stable long-chain tethering crosslinks. The sacrificial crosslinks may be chosen from any easily hydrolyzed crosslinks known to one of skill in the art of polymer science such as, for example, crosslinks containing an ester functionality. The chemically stable crosslinks may be chose from any such crosslinks known to one of skill in the art of polymer science such as, for example, crosslinks containing an amide functionality.

Dependent upon the chemical sensitivity of the molecular recognition agent suspended in the hydrogel layer 24, the hydrogel layer 24 is attached to the tethering hydrogel layer 23. However, if the molecular recognition agents are sensitive to high or low pH, the sacrificial crosslinks may be removed first and then the hydrogel layer 24 may be attached to the tethering hydrogel layer 23. The hydrogel layer 24 is attached such that the 2DPC array 25 forms a monolayer.

The molecular recognition agent 13 may be any be any analytical tool known to one of skill in the art for detecting an analyte in a bodily fluid such as, for example, a molecular recognition agent, an analyte imprinted sensor or a monoclonal antibody. Examples of molecular recognition agents include crown ethers, calixarenes, rotaxanes, etc. The 2DPC sensors according to the present disclosure may preferably be used for applications for clinical chemistry point-of-care sensing that would be used either as a multianalyte sensor array in the bottom of shallow multiwell plates, or as a microfluidic point-of-care 2DPC sensor array for multiple analytes.

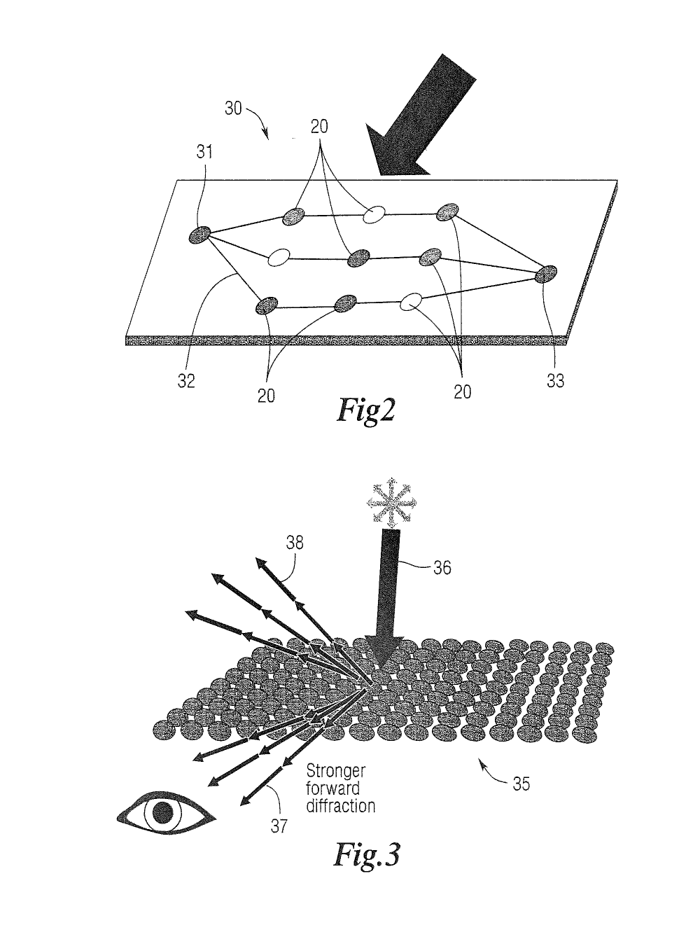

The chemical sensors 20 described in the present application may be designed to analyze for one substance or for multiple analytes simultaneously. FIG. 2 illustrates a point-of-care simultaneous sensing device 30. Device 30, comprises and inlet 31, a wick 32, chemical sensors 20 and an outlet 33. It is envisioned that the sensing device 30 may have an inlet 31 for the sample or bodily fluid to be analyzed and a wick 32 for transporting the sample or bodily fluid to the membrane filter layer 21. Suitable wick for sensing devices 30 of the present application may be chosen from any known to one of skill in the art such as, for example, microfluidic channels. If the chemical sensor 20 of the present application is designed to analyze for multiple analytes, it is envisioned that the sensing device 30 may have an inlet 31 for the bodily fluid and multiple wick 32, such as, for example, microfluidic channels, leading to each membrane filter layer 21. Thus, the sensing devices 30 of the present application can be visualized as small, self-contained analytical devices for point-of-care service even in low resource environment situations.

One advantage of sensing device 30 of the present disclosure is the provision of a point-of-care clinical sensing device that performs clinical chemistry measurements from fingerstick blood drawn. The microfluidics based-design of sensor 30 of FIG. 2 uses fingerstick blood placed on the inlet 31 is quickly wicked to a broad array of diagnostic 2DPCs 20. The 2DPC sensor diffraction wavelengths will, within a short time of few minutes or less, report on concentrations of analytes by individually changing their diffraction colors. The analyte determination can be read out visually with the aid of a color chart, or the readout can be made with the aid of a simple camera-cell phone-type device with the results easily downloaded into the patient's record.

The sensitivity and utility of the novel 2DPC array nanoscale hydrogel sensing materials of the present disclosure will be much higher than that of the 3DPC hydrogel sensor materials previously pioneered. The diffraction shifts monitored result from 2DPC array area changes of ˜500 nm thick 2DPC hydrogels. This hydrogel volume is relatively unconstrained by the third dimension that gently tethers the 2DPC array hydrogel to the substrate. Because the third tethering dimension does not significantly constrain the 2DPC array hydrogel volume, unprecedented large hydrogel 2DPC array area changes can occur.

The 2DPC array sensor of the present disclosure also show novel 2D array diffraction phenomena that give rise to extraordinary high SN ratios for analyte measurements. The 2D diffraction phenomena differ dramatically from that of 3D arrays. 2DPC diffraction gives rise to dispersion of the incident light that is diffracted by the 2DPC array lattice (indicated as 35 in FIGS. 3 and 25 in FIG. 6). Much more light is diffracted by the 2DPC array than from 3DPC made from the same nanoparticles with similar particle spacings. FIG. 3 illustrates a 2DPC array 35 of the present disclosure, wherein a greater proportion of the incident light 36 is forwarded diffracted 37 rather than back diffracted 38.

Instead of the 3DPC diffracting only a defined narrow wavelength band when the 3DPC reciprocal lattice vector meets the Bragg diffraction condition, the 2DPC array reciprocal lattice vector simultaneously diffracts a large range of wavelengths over the 2DPC diffraction-defined range of diffraction angles (FIG. 3). This enables monitoring of the entire diffraction spectrum to relate the diffraction wavelength to angle in order to determine analyte concentrations. These measurements benefit from the much higher light intensities.

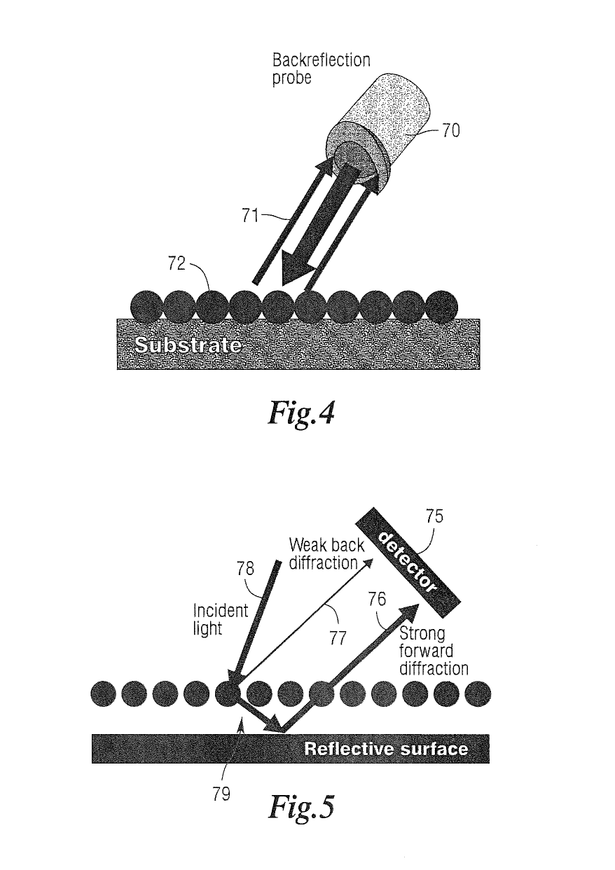

Thus, preferred embodiments of the present disclosure using this 2DPC diffraction phenomenon simplify the diffraction measurements by allowing for the use of a backreflection fiber optic probe (in the Littrow configuration) to determine the 2D array spacing. FIG. 4 illustrates the use of a backreflection probe 70 to measure the diffracted light 71 from a 2DPC array sensor 72. Alternatively, a single wavelength beam could be used to measure the diffraction angle relative to that of the incident light.

This contrasts dramatically with measurements of the more challenging fcc 3D array diffraction. For the 3D case, backscattering diffraction only occurs for light incident along the fcc array 111 direction. Less conveniently the diffraction can be measured by monitoring the diffracted light along the reflection direction (at the complement to the incident light angle relative to the normal to the 111 planes). A single wavelength of light cannot easily be used to monitor the 3D array spacing because only a single angle exists where this particular wavelength diffracts.

Preferred 2DPC ultrathin sensor arrays according to the present disclosure (indicated as 35 in FIGS. 3 and 25 in FIG. 6) show amazingly intense diffraction efficiencies for forward diffraction such that the diffracted color can be visually monitored to determine analyte concentrations. Such 2DPC arrays diffract over 80% of the incident light and about 90% of this diffracted light is concentrated into the forward direction as shown in FIG. 3. Further, a model of the present disclosure explains these results. The model utilizes the Mie scattering relations to predict the angular dependence of the diffraction efficiencies from the particles of the 2DPC array. The 2DPC diffraction wavelength angular dependence surprisingly does not depend on the 2DPC array refractive index if the 2DPC array is parallel to the interface. In contrast, the diffracted intensities depend upon the refractive index due to refraction of the incident and diffracted light. The 2DPC diffraction is ultrabright and easily monitored. It is also important to understand that the backward diffracted light, while less bright, is still clearly evident and useful. It contains 5% of the incident intensity and can easily be used to readout the analyte concentration. FIG. 5 illustrates the use of a detector 75 to measure both the forward diffracted light 76 and back diffracted light 77 diffracted from incident light 78 hitting a preferred 2DPC sensor 79 according to the present disclosure.

Another innovation of the 2DPC sensors of the present disclosure is that the ultrathin 2DPC array (500 nm) film enables selective and sensitive sensing of macromolecular species bound to the surface of the 2DPC. Changes in hydrogel surface bound charge or surface free energy of mixing or surface crosslinking will drive 2DPC array spacing changes. Thus, the sensing materials of the present disclosure are useful in applications for sensing analytes diffusing from tissue surfaces and to sense proteins and other species bound to tissue and cell surfaces.

The 2DPC sensors of the present disclosure are also innovative in their use of hydrogel imprinting for analyte recognition and hydrogel volume phase transitions. The literature indicates that imprinting requires hard polymer or inorganic materials. However, based on recent studies that indicated successful analyte imprinting of high concentration hydrogels, it has been found that the lower concentration hydrogels used in 3DPCs can be successfully imprinted as well. Preferably, such imprinting process is applied to the low polymer concentration 2DPC arrays of the present disclosure to make them highly selective and sensitive to any analyte desired.

Another aspect of the present application involves a method for analyzing, a bodily fluid utilizing chemical sensors 20. Often the diagnosis and treatment of disease requires determination of chemical species diagnostic of a patient's condition. The methods for analyzing a bodily fluid, disclosed in the present application, utilize novel chemical sensors 20 containing novel nanoscale, two dimensional photonic crystals (2DPC) in a chemically-sensitive hydrogel 24. The methods for analyzing a bodily fluid of the present application provide point-of-care analyses and can be used in low resource environment situations.

The methods of the present application offer many advantages over standard in vitro chemical analyses performed in a central clinical laboratory. First, the present methods offer diagnostic point-of-care patient analysis. Second, the present methods can be performed with a minimal amount of bodily fluid such as, for example, a fingerstick drop of blood. Third, the present methods can be performed in low resource environment situations such as, for example, first responders, aid workers, military medics, etc.

To practice the methods of the present disclosure, a sample of the bodily fluid to be analyzed is obtained, such as, for example, blood by a fingerstick. The sample is placed onto the membrane filter layer 21 of the chemical sensor 20 of the present application either directly or indirectly. If the chemical sensor 20 is configured to receive the sample indirectly, a wick 32 such as, for example, a microfluidic channel preferably is present to convey the sample to the membrane filter layer. Once the sample has reached the membrane filter layer 21 it will pass through the membrane filter layer 21 and subsequently pass through the mirror 22 and tethering layers 23. Upon reaching the hydrogel layer 24 the sample can interact with the molecular recognition agent. The interaction between the analyte 14 and molecular recognition agent 13 will cause the hydrogel layer 24 to either swell or shrink thus changing the distance of the 2DPC layer from the mirror layer 22 versus a control. Thus, a color change can be observed for light shone through the hydrogel layer 24 that comprises molecular recognition agent and a 2DPC self-assembling array onto the mirror layer 22 that is diffracted through the 2DPC self-assembling array and compare this color change to a color chart.

The interaction of the analyte 14 with the molecular recognition agent can be observed by utilizing the properties of the novel two-dimensional photonic crystals (2DPC) described in this application. The chemically sensitive hydrogel layers 24 that comprises molecular recognition agent and a 2DPC self-assembling array are easily fabricated from preassembled 2D nonoparticle arrays to which are polymerized the molecular recognition agent containing hydrogels. The 2DPC of the present application have been chosen because they easily self assemble and in combination with a reflective surface diffract more than 50% of the incident light making the diffraction color visually vivid.

The 2DPC hydrogel sensors of the present disclosure preferably are made ultrasensitive by utilizing thin sensing hydrogels with very low crosslink densities. The sensing hydrogel volume phase transitions are minimally constrained by the attachment to the substrate. This is accomplished by decoupling the sensing hydrogel volume changes from that of the hydrogel that tethers the 2DPC array sensing hydrogel to the mirror substrate. This enables the extraordinarily large 2DPC lattice expansions and contractions required for ultrahigh detection sensitivities.

For simple point-of-care sensing applications, the 2DPCs of the present disclosure preferably are attached onto Al or Ag mirrors (FIGS. 5 and 6) so that the forward diffracted light is captured by the light sensing detector 75. The 2DPC will be attached to the front surface mirrors by utilizing tethering hydrogels that contain a low density of long-chain crosslinkers. This is accomplished by functionalizing the mirror surface with vinyl groups and then polymerizing a strong hydrogel sheet with a high density of sacrificial, easily broken crosslinks and a lower density of chemically stable long-chain tethering crosslinks. The tethering hydrogel preferably are synthesized mainly with crosslink functionalities such as esters, that are easily hydrolyzed, but the hydrogel will also contain a low density of hydrolysis resistant long chain PEG-type crosslinkers that utilize amide functionalities, for example.

Ester crosslinks may be easily hydrolyzed at high pH in the presence of TEMED. The tethering hydrogel must be initially relatively rigid so that the sensing 2DPC-hydrogel can be attached onto it. Preferably, the hydrogel-mirror substrate is polymerized onto the 2DPC using a hydrogel polymerization solution containing a low concentration of an (almost) nonhydrolyzable crosslinker like bisacrylamide. This will form the 2DPC-hydrolyzable hydrogel-min- or sandwich. The tethering hydrogel in base and TEMED is then hydrolyzed to remove all but the low concentration non-hydrolyzable tethering hydrogel crosslinks.

The molecular recognition agents 13 are preferably attached during polymerization of the sensing 2DPC hydrogel, or if the recognition chemistry is sensitive to pH, the 2DPC array sensing hydrogel will be functionalized after hydrolyzing the sacrificial crosslinks

The complete 2DPC sensor shows extraordinary sensing sensitivities since the modulus of the sensing hydrogel is minimized by its low crosslink density. This 2DPC operates well for detection of analytes in low volume solutions that will flow onto the 2DPC array surface (FIG. 6). The incident light will be diffracted to, for example, the fiber optic reflection probe (FIG. 4). The wavelength of the diffracted light into the reflection probe will indicate the array spacing. The analyte concentration will be determined from the previously determined sensor calibration.

The 2DPC sensors of the present disclosure preferably, in many applications, will utilize small volumes of fingerstick blood whose absorption prevents transmission of the incident and diffracted light. In this case, a preferred 2DPC is constructed where the blood is introduced through the 2DPC mirror. The mirror, in this case, is preferably evaporated onto a flat surface mesh screen that contains numerous small channels. The exterior of the mesh-mirror is preferably attached to a membrane filter to remove the red blood cells, leaving only clear liquid plasma to interact with the 2DPC sensing hydrogel (FIG. 6). The mesh mirror surface is preferably continuously covered by the tethering hydrogel that attaches the sensing hyrdrogel. In sensing cases where large blood volumes are needed, the blood cells preferably are filtered out prior to contact with the 2DPC.

The 2DPC sensors of the present disclosure can be read out visually, by using a light source (ambient or artificial) and eyepiece that defines the Littrow angle for the incident and diffracted light. The analyte concentrations are determined by using calibrated color charts. However, most applications will directly measure the diffracted wavelength.

For point-of-care analyte determinations in the field, an inexpensive color CCD camera/light source is preferred to record the diffracted angular wavelength dependence using a software program to analyze the photograph with stored calibration data. This is straightforward since the color at any Littrow angle results from a small range of wavelengths (FIG. 8); the color recorded in the small angular width collected by the camera will translate to the analyte concentration through the calibration. In cases where the time dependence of the diffracted wavelength is analyzed, a series of photographs are preferably monitored that record the changing diffraction colors and the concentration then calculated from the diffraction time evolution. A battery powered light source may be used as well.

It is recognized that variations in the clinical state of a patient may result in differences in the composition of the samples in addition to differences in the analyte concentration. For example, patients with extreme states of hyper or hyponatremia, or hyper or hypokalemia and other extreme deviations from normal physiology may impact the response of these 2DPC array sensors. Thus, the response of the 2DPC array sensors of the present disclosure are carefully characterized in these types of situations, as discussed more fully herein.

The 2DPC sensors of the present disclosure preferably are calibrated by measuring the 2DPC response in aqueous solutions with ionic strengths and compositions similar to blood. The angular dependence of the diffracted wavelengths as a function analyte concentration is then monitored, as is the impact of pH, ionic strength and buffer concentration on the response to the analytes. This will determine the degree to which constant ionic strength, pH and buffering capacity will need to be ensured for clinical samples.

Preferably, such same studies will be repeated in blood from normal individuals by adding varying concentrations of analytes to determine the analytical sensitivities, detection limits and precision. For most cases it is expected that whole blood can be used by completing the measurements prior to clotting. If necessary, blood studies will be completed after addition of EDTA or heparin. The objective is to accomplish these measurements with low blood volumes, preferably obtained by fingersticks. It should be noted that the 3DPC creatinine sensor operates well in anticoagulated blood.

FIG. 8 illustrates an analytical kit 40 of the present disclosure for analyzing bodily fluids. Analytical kit 40, comprises a 2DPC array chemical sensor 20, a CCD camera/light source combination 41 and diagnostic software that measures and analyzes the diffracted light from 2DPC array 25.

### EXAMPLES

### Example 1

Preferably, a 2DPC array chemical sensor for use in analyzing a sample or bodily fluid may be prepared by the following steps:
a) obtain a suitable membrane filter layer;
b) chemically attach a mirror layer to the membrane filter layer:
c) preparing the mirror layer for polymerization;
d) polymerizing a tethering hydrogel layer, with a high density of easily broken crosslinks and a lower density of chemically stable long-chain tethering crosslinks, onto the prepared mirror layer;
e) polymerize a hydrogel layer, comprising molecular recognition agent and a 2DPC self-assembling array onto the tethering hydrogel layer; and
f) hydrolyze the easily broken crosslinks in the tethering hydrogel layer.

### Example 2

Preferably, a 2DPC array chemical sensor for use in analyzing a sample or bodily fluid may be prepared by the following steps:
a) obtain a suitable membrane filter layer;
b) chemically attach a mirror layer to the membrane filter layer:
c) preparing the mirror layer for polymerization;
d) polymerizing a tethering hydrogel layer, with a high density of easily broken crosslinks and a lower density of chemically stable long-chain tethering crosslinks, onto the prepared mirror layer;
e) hydrolyze the easily broken crosslinks in the tethering hydrogel layer; and
f) polymerize a hydrogel layer, comprising molecular recognition agent and a 2DPC self-assembling array onto the tethering hydrogel layer.

### Example 3

Preferably, a sample or bodily fluid may be analyzed by the following steps:
a) obtaining a sample or bodily fluid;
b) placing an amount of the sample or bodily fluid onto a chemical sensor, comprising:
   1) a hydrogel layer, comprising a molecular recognition agent and a 2DPC self-assembling array;
   2) a tethering hydrogel layer;
   3) a mirror layer; and
   4) a membrane filter layer,

wherein, the layers are arranged such that an amount of the sample or bodily fluid to be analyzed may be placed on an exposed surface of the membrane filter layer, travel through the membrane filter layer, subsequently travel through the mirror layer and then subsequently travel through the tethering hydrogel layer to the hydrogel layer,
c) allow the bodily fluid to interact with the hydrogel, comprising molecular recognition agent and a 2DPC hydrogel self-assembling array; and
d) allowing light to pass through the hydrogel, comprising molecular recognition agent and a 2DPC self-assembling array onto the mirror layer and observing the change in diffraction versus a control.

It should be understood that while this invention has been described herein in terms of specific embodiments set forth in detail, such embodiments are presented by way of illustration of the general principles of the invention, and the invention is not necessarily limited thereto. Certain modifications and variations in any given material, process step or chemical formula will be readily apparent to those skilled in the art without departing from the true spirit and scope of the present invention, and all such modifications and variations should be considered within the scope of the claims that follow.

What is claimed is:

1. A chemical sensor comprising:
a) a hydrogel layer, comprising one or more molecular recognition agents and a 2DPC self-assembling array; and
b) a mirror layer.

2. The chemical sensor according to claim 1, further comprising a tethering hydrogel layer.

3. The chemical sensor according to claim 2, further comprising a membrane filter layer.

4. The chemical sensor according to claim 3 wherein, the layers are arranged such that an amount of a sample or bodily fluid to be analyzed may be placed on an exposed surface of the membrane filter layer, travel through the membrane filter layer, the mirror layer and the tethering hydrogel layer to the hydrogel layer.

5. The chemical sensor according to claim 1, wherein the hydrogel layer has an imprinted analyte well.

6. The chemical sensor according to claim 1, wherein the hydrogel layer is formulated to perform an analysis selected from the group consisting of: blood osmolality quantitation; blood pH determination; cation detection and/or quantitation; anion detection and/or quantitation; ammonia detection and/or quantitation, metal detection and/or quantitation; urea detection and/or quantitation; uric acid detection and/or quantitation; protein detection and/or quantitation; and cell and tissue surface chemical detection and/or quantitation.

7. The chemical sensor according to claim 1, wherein the hydrogel layer contains an antibody or a monoclonal antibody.

8. The chemical sensor according to claim 6, wherein the cation to be detected and/or quantified is selected from the group consisting of: sodium, potassium, calcium, magnesium, ammonium and zinc.

9. The chemical sensor according to claim 6, wherein the anion to be detected and/or quantified is selected from the group consisting of: chloride, phosphate and bicarbonate.

10. The chemical sensor according to claim 6, wherein the metal to be detected and/or quantified is mercury.

11. The chemical sensor according to claim 3, further comprising a wick for conveying the sample or bodily fluid to a plurality of different molecular recognition agents in the hydrogel layer.

12. A process for the preparation of a chemical sensor having
a hydrogel layer, comprising a molecular recognition agent and a 2DPC self-assembling array;
a tethering hydrogel layer;
a mirror layer; and
a membrane filter layer,
comprising:
chemically attaching the mirror layer to the membrane filter layer;
preparing the mirror layer for polymerization;
polymerizing the tethering hydrogel layer, with a high density of easily broken crosslinks and a lower density of chemically stable long-chain tethering crosslinks, onto the prepared mirror layer;
polymerizing a hydrogel layer, comprising molecular recognition agent and a 2DPC self-assembling array onto the tethering hydrogel layer; and
hydrolyzing the easily broken crosslinks in the tethering hydrogel layer.

13. The process for the preparation of a chemical sensor according to claim 12 wherein the polymerizing and hydrolyzing are reversed in order of performance.

14. The process for the preparation of a chemical sensor according to claim 12, wherein the mirror layer is prepared with one or more vinyl groups.

15. The process for the preparation of a chemical sensor according to claim 12, wherein the easily broken crosslinks have an ester functionality and wherein the ester functionalities are hydrolyzed under basic conditions in the presence of tetramethyehtylenediamine (TEMED).

16. The process for the preparation of a chemical sensor according to claim 12, wherein the chemically-stable long-chain tethering crosslinks have an amide functionality.

17. The process for the preparation of a chemical sensor according to claim 12, wherein the hydrogel layer, comprising a molecular recognition agent and a 2DPC self-assembling array is less than about 1 μM thick.

18. A method for analyzing a sample or bodily fluid, comprising:
obtaining a sample or bodily fluid;
placing an amount of the sample or bodily fluid onto a chemical sensor, comprising:
a hydrogel layer, comprising a molecular recognition agent and a 2DPC self-assembling array;
a tethering hydrogel layer;
a mirror layer; and
a membrane filter layer,
allowing the bodily fluid to interact with the hydrogel layer; and
allowing ambient or artificial light to pass through the hydrogel layer onto the mirror layer and
observing a change in diffraction versus a control.

19. A chemical sensor comprising:
a) an ultrathin hydrogel layer, comprising an imprinted analyte sensor and/or a molecular recognition agent; and
b) a mirror layer;
wherein the hydrogel layer diffracts about 80% of incident light.

20. The chemical sensor according to claim 19, wherein about 90% of incident light diffracted by the hydrogel layer is forward diffracted.
